(19) United States
(12) Patent Application Publication
(10) Pub. No.: US 2002/0000225 A1
(43) Pub. Date: Jan. 3, 2002

(54) Lockout mechanism for aerosol drug delivery devices

(76) Inventors: Carlos Schuler, Cupertino, CA (US); Normand Nantel, San Carlos, CA (US); Thomas Tarara, Burlingame, CA (US); Jeff Weers, Half Moon Bay, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/852,408

(22) Filed: May 9, 2001

Related U.S. Application Data
(60) Provisional application No. 60/208,896, filed on Jun. 2, 2000.

(51) Int. Cl. A61M 11/00; A61M 15/00; A61M 16/10

(52) U.S. Cl. 128/200.14; 128/203.12

(57) ABSTRACT

A hand-held, portable, aerosol drug delivery system comprises a disposable container containing a drug formulation. The system also includes an aerosol generator for aerosolizing the drug formulation, and a prevention device. The prevention device prevents access to the drug formulation when in an inactive state and permits access to the drug formulation when in an activated state.

### CROSS REFERENCE TO RELATED APPLICATIONS

This application is a continuation in part and claims the benefit of U.S. Provisional Patent Application No. 60/208,896, filed Jun. 2, 2000, the complete disclosure of which is herein incorporated by reference.

### FIELD OF THE INVENTION

The present invention relates generally to a lockout mechanism for use with various aerosol drug delivery devices. In particular, the present invention provides a lockout mechanism for preventing dosing of the device outside of a certain dosing regimen. The lockout mechanism allows access to the drug formulation when placed in an activated state where the lockout mechanism is disengaged and prevents access to the drug formulation when in an inactive state where the lockout mechanism is engaged, thereby significantly reducing power requirements.

### BACKGROUND OF THE INVENTION

It is often desirable to control access to a drug contained within a drug delivery device in order to safeguard against accidental or unintended dosing. This is particularly the case where the drug may be harmful to an unintended user, such as a child, or when the drug is potentially toxic to an unintended user, as in the case of narcotic drugs. As a result, it is known in the art to provide drug delivery devices with a variety of safety mechanisms to control access to the drug contained within such devices.

One example of such a safety mechanism is common in the field of patient-controlled analgesia (PCA). PCA is an effective means of postoperative pain management wherein on-demand doses of a narcotic are coupled with a baseline infusion of the narcotic in order to treat breakthrough pain when experienced by the patient. PCA systems utilize an intravenous infusion system which includes a microprocessor for monitoring the frequency of on-demand dosing requests and for determining whether it is safe to administer the next dose as demanded by the patient. PCA devices therefore monitor administration of the narcotic drug according to a safe dosing regimen and prevent the administration of doses outside of the determined safe range.

Another type of device where control of the drug may be important is with devices that are employed to aerosolize a drug formulation, either in the form of a powder or a liquid. Examples of dry powder dispersion devices are described in U.S. Pat. Nos. 5,458,135, 5,775,320, 5,740,794 and 5,785,049, and in co-pending U.S. patent application Ser. Nos. 09/004,558, filed Jan. 8, 1998; 09/312,434, filed Jun. 4, 1999; 60/136,519, filed May 28, 1999, and 60/141,793, filed Jun. 30, 1999, the complete disclosures of which are herein incorporated by reference.

Examples of liquid aerosolization devices include metered dose inhalers, nebulizers and the like. Lockout mechanisms have been provided for aerosolization devices used for administering narcotic drugs, such as disclosed in U.S. Pat. Nos. 5,724,957 and 5,910,301, incorporated herein by reference. Additionally, a lockout device for controlled release of a drug from a patient-activated dispenser is disclosed in U.S. Pat. Nos. 5,507,277, 5,694,919 and 5,735,263, incorporated herein by reference.

One important issue to consider when providing various safety features for drug delivery devices is the need to provide such features in a cost-effective manner. This is particularly true for systems where some or all of the components are disposable.

Hence, the invention is related to techniques for controlling access to a drug that is to be administered to a patient, particularly with aerosolization devices where a drug is aerosolized prior to delivery. The invention is also related to cost saving techniques so that such devices may be constructed in a cost effective manner.

### SUMMARY OF THE INVENTION

In one embodiment, the invention provides an inexpensive aerosol drug delivery device that is to be used with a disposable container containing a drug formulation. The delivery device includes an aerosol generator to aerosolize an amount of the drug formulation from the container. A prevention device is further provided to prevent administration of the drug formulation when in an inactive state and to permit administration of the drug formulation when in an activated state. As used herein, “inactive state” refers to a condition where no current is flowing through the lockout mechanism, and the lockout mechanism is engaged so as to prevent the administration of a dose. As used herein, “active state”, refers to a condition where current is flowing through the lockout mechanism which is in a disengaged position allowing dosing. This arrangement allows for a more effective usage of the power requirements necessary to permit administration, thereby providing a more cost-effective device.

In one aspect, the prevention device comprises an electronic lockout device having a lockout element that is positioned in a dose preventing position when in the inactive state, and is movable to a dosing permitting position when electric current is supplied to place the lockout device in the activated state. In this way, current is only needed at the time of dosing, thereby greatly minimizing the power needed to operate the device. Conveniently, the lockout device may include circuitry for supplying electrical current to move the lockout element to the dose permitting position when the lockout device is in the activated state. A controller having an associated memory for storing a dosing condition may also be provided. The controller may then be employed to send a signal to place the lockout device in the activated state only after the dosing condition has been satisfied. For example, the controller may receive information from an electronic clock to determine when a certain amount of time has elapsed since the previous dose. Once this condition has been satisfied, the controller may send a signal to activate the lockout device when a request for dosing is received.

In one specific aspect, the container comprises a canister, and the aerosol generator comprises a metering valve and an actuator operably coupled to the canister. The device may further include a housing, with the canister being reciprocally held within at least a portion of the housing. The canister may be moved between a home position and a dosing position where the actuator is engaged to open the metering valve and to permit the escape of a metered amount of the drug formulation from the canister. With such a configuration, the lockout element may be positioned to prevent engagement of the actuator when in the dose preventing position to thereby prevent opening of the metering valve. In one aspect, the lockout element has a distal end that is engageable with the canister to prevent substantial displacement of the canister into the housing when the lockout element is in the dose preventing position. Upon placement of the preventing device into the activated state, the distal end of the lockout element is retracted to permit displacement of the canister into the housing and to permit engagement of the actuator to open the metering valve.

In an alternative arrangement, the canister may be movable within the housing when the preventing device is in the inactive state. With such a configuration, a stop may be reciprocally disposed within the housing below the actuator. Further, the lockout element may have a distal end that is engageable with the stop when in the activated state to prevent movement of the stop within the housing. In this way, displacement of the canister engages the actuator with the stop to permit dispensing of the metered drug formulation when the preventing device is in the activated state. When inactive, the canister may still be moved within the housing, but dosing will not occur.

Optionally, a high pressure gas source may be provided to assist in aerosolizing the drug formulation when the preventing device is in the activated state. Alternatively, the device may be a breath actuated device that relies upon patient inhalation.

In another aspect, a dose counter may be provided to count the number of doses of the drug formulation dispensed from the container. For example, the dose counter may comprise a dose counting circuit that is positioned to sense when the container has been reciprocated within the housing. Conveniently, a display may be provided to indicate if the container contains an amount of drug formulation.

In another particular aspect, a nozzle may be coupled to the canister, and a mouthpiece may be disposed to receive the drug formulation from the nozzle. In one aspect, the mouthpiece has a first end and a second end, and the nozzle is positionable within an opening adjacent the first end of the mouthpiece to permit the aerosolized drug formulation to be delivered to a patient upon inhalation through the second end of the mouthpiece.

The invention further provides an exemplary method for administering a drug formulation. According to the method, a container is provided having an amount of a drug formulation along with an aerosol generator to aerosolize the drug formulation. Transfer of the drug formulation from the container and to the aerosol generator is prevented with an electronic lockout device when the lockout device is in an inactive state. When ready to aerosolize an amount of the drug formulation, electrical current is supplied to the lockout device to place the lockout device in an active state, thereby permitting the transfer of the drug formulation from the container and to the aerosol generator.

In one aspect, the electronic lockout device comprises a lockout element that is positioned in a dose preventing position when in the inactive state. The lockout element is moved to a dosing permitting position when electric current is supplied to place the lockout device in the activated state. In another aspect, the container comprises a canister having a metering valve and an actuator, and the canister is reciprocally held within a housing. The canister is depressed into the housing to a dosing position to engage the actuator and to release a metered amount of the drug formulation when the lockout device is in the active state. When the lockout element is in the dose preventing position, engagement of the actuator is prevented.

In one aspect, the canister is engaged with the lockout element to prevent movement of the canister to the dispensing position when the lockout element is in the dose preventing position. Upon supply of electrical current, the lockout element is disengaged from the canister to permit movement of the canister to the dispensing position upon supply of the electrical current. Alternatively, the lockout element is engaged with a stop that is positioned below the actuator upon supply of the electrical current. In this way, the canister may be depressed into the housing to engage the actuator with the stop.

In another aspect, the supply of the electric current to the lockout device is stopped after the drug formulation has been transferred from the container to reduce the amount of power usage. In one particular aspect, electric current is supplied to the lockout device to permit another dosing only after a certain dosing conditions have been satisfied. Optionally, the number of doses transferred from the container may be counted to determine when all of the drug formation has been dispensed. A display may then be provided to indicated whether the container contains an amount of drug formulation.

In another embodiment, the invention provides a hand-held, portable, aerosol drug delivery system that comprises a housing having a mouthpiece and a canister that is movable within the housing when manually depressed into the housing. The canister has a metering valve that is operable to release a metered amount of a drug formulation from the canister. A control system is also provided to control opening of the valve such that the valve is only opened when a force is manually applied to depress the canister into the housing and when a dosing condition has been satisfied.

In one aspect, the control system comprises a controller and a locking mechanism, and the controller is configured to send a signal to the locking mechanism to permit opening of the valve once the dosing condition has been satisfied. For example, the dosing condition may be the passage of a certain amount of time between dosings. Further, an electronic clock may be coupled to the controller to measure the passage of time between dosings.

In another aspect, the locking mechanism is normally in a dose preventing position and is movable to a dosing position when electrical current is supplied to the locking mechanism to permit opening of the valve when the canister is depressed. As one example, the locking mechanism may include a locking element that engages the canister to prevent depression of the canister into the housing when in the dose preventing position. As another example, the canister may include an actuator, and the locking element may be configured to engage a stop that in turn engages the actuator when in the dose permitting position and when the canister is depressed into the housing.

In another aspect, the invention provides a safe and effective aerosol delivery system for the administration of nicotine for smoking cessation therapy.

These and other aspects of the present invention will be apparent in view of the figures and detailed description that follows.

### BRIEF DESCRIPTION OF THE DRAWINGS

FIG. 1 is a flow chart illustrating one method for the pulmonary delivery of a drug formulation according to the invention.

FIG. 2 is a perspective view of one embodiment of an aerosol drug delivery system according to the invention.

FIG. 3 is a schematic view of an electronic lockout device that may be used to control drug aerosolization according to the invention.

FIGS. 3A and 3B illustrate the manner of operation of the lockout device of FIG. 3.

FIG. 4 is a schematic view of an alternative lockout device according to the invention.

FIGS. 5A and 5B schematically illustrate operation of still another embodiment of a lockout device according to the invention.

FIG. 6 schematically illustrates a canister that is held within a housing according to the invention.

FIG. 7 schematically illustrates a capacitive detection system for detecting canister actuation according to the invention.

FIG. 8 schematically illustrates a membrane switch for detecting canister actuation according to the invention.

FIG. 9 schematically illustrates a touch-sensitive circuit for detecting canister actuation according to the invention.

FIG. 10 illustrates an optical detection system for detecting canister actuation according to the invention.

FIG. 11 schematically illustrates a magnetic detection system for detecting canister actuation according to the invention.

FIG. 12 schematically illustrates a pressure detection system for detecting canister actuation according to the invention.

FIG. 13 schematically illustrates a piezoelectric film employed to detect canister actuation according to the invention.

FIG. 14 schematically illustrates an optical detecting system for detecting emission of a dose according to the invention.

FIG. 15 schematically illustrates an acoustic detection system for detecting emission of a dose according to the invention.

FIGS. 16A and 16B schematically illustrate one embodiment of a lockout mechanism according to the invention.

FIGS. 17A and 17B schematically illustrate an alternative lockout mechanism according to the invention.

### DESCRIPTION OF THE SPECIFIC EMBODIMENTS

The invention provides for the controlled delivery of a drug formulation according to a certain dosing regimen. In this way, the drug formulation is only delivered after certain dosing conditions have been satisfied. For example, a patient may be prescribed a certain dosing regimen where the drug is to be delivered in specified time intervals, e.g. three administrations over a ten minute interval. As another example, the patient may be limited to a certain number of doses in a 24 hour period. The invention provides techniques for preventing the delivery of the drug formulation until all dosing conditions have been satisfied. In this manner, the patient is able to safely follow a certain dosing regimen, with safeguards in place to prevent overdosing.

The dosing conditions specified by the dosing regimen may be electronically programmed into a drug delivery device having a controller that is able to control a lockout device based on the dosing conditions. In this way, access to the drug formulation is prevented by the lockout device until the appropriate conditions have been satisfied. At such a point, the controller may send the appropriate signals to operate the lockout device and permit dosing to occur.

The techniques of the invention may be used with a wide variety of drug delivery devices, and will be particularly useful with devices that deliver the drug formulation to the respiratory system. For example, the invention may be used with portable, hand-held pulmonary delivery devices. Specific examples of devices that may be used with the invention include metered dose inhalers (MDIs) such as that disclosed in U.S. Pat. No. 4,955,371, the disclosure of which is herein incorporated by reference. Formulations suitable for administration with MDIs and their preparation are described in copending U.S. application Ser. No. 09/218,212, filed Dec. 22, 1998, the complete disclosure of which is herein incorporated by reference. As a further example, the invention may be used with powder dispersion devices for delivering an aerosolized powder to the lungs. Exemplary powder dispersion devices are described in, for example, U.S. Pat. Nos.5,785,049 and 5,740,794, and in U.S. application Ser. Nos. 09/004,558 and 60/141,793, the complete disclosures of which are herein incorporated by reference. The drug delivery devices may employ a pressurized gas source to assist in aerosolizing and delivering the drug formulation to the patient. Alternatively, such devices may be breath activated devices which rely on the patient's own inhalation for aerosolization and delivery. With such devices, the invention may be configured to prevent opening of a receptacle containing the drug formulation.

The invention may be used with essentially any type of drug formulation that is capable of pulmonary delivery, including drug formulation in both liquid form and dry powder form. The invention will find particular use when the drug formulation is a narcotic or other controlled substance where administration needs to be closely controlled to prevent overdosing or misuse. According to one preferred embodiment, the present invention provides a safe and effective treatment for smoking cessation by administering nicotine from an aerosol delivery system. Optionally, the devices of the invention may further include various child lockout features to prevent any access by a child.

The drug delivery devices of the invention may conveniently use various indicators and/or buttons to indicate when dosing is permitted, i.e. when a dose has been qualified. For example, a status button may be provided to enter a request for dosing. If dosing is permitted, a certain visual and/or audio indicator may be actuated. Another indicator may be actuated if dosing is not permitted. As another example, the devices may include a dose counter to count the number of doses administered. In this way, a display may be provided indicating when all of the drug formulation has been dispensed.

To control dispensing of the drug formulation, an electronic lockout system may be utilized. The lockout system may be configured to prevent access to the drug formulation when in an inactive state, i.e. when no power is being supplied. To permit access to the drug formulation, electrical power is supplied to the system to place the system in an active state. In this way, the operational power requirements may be minimized. Further, such a design permits the device to be constructed in a cost efficient manner and to be both portable and disposable.

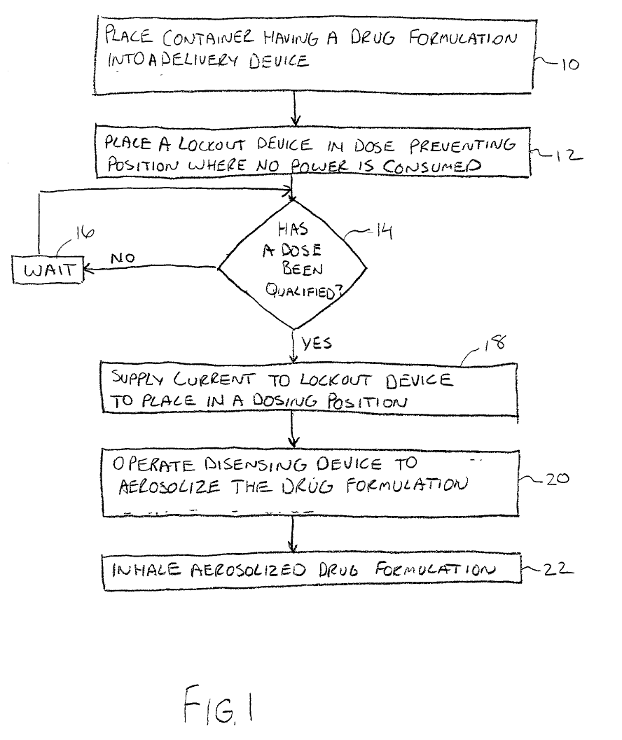

Referring now to FIG. 1, one method for the controlled dispensing of a drug formulation will be described. As shown in step 10, a container having a drug formulation is placed into a delivery device. When the container is within the delivery device, a lockout device is normally in a dose preventing position to prevent dispensing of the drug formulation as shown in step 12. When in the dose preventing position, no power is consumed to minimize power usage. The method then proceeds to step 14 where a determination is made as to whether a dose has been qualified, i.e. whether certain dosing conditions have been satisfied. If not, the user must wait as shown in step 16 until an appropriate amount of time has passed. Once the dose has been qualified, electrical current is supplied to the lockout device to place the lockout device in a dosing position as shown in step 18. The delivery device may then be operated to aerosolize the drug formulation as shown in step 20. The aerosolized drug formulation may then be inhaled by the patient as shown in step 22.

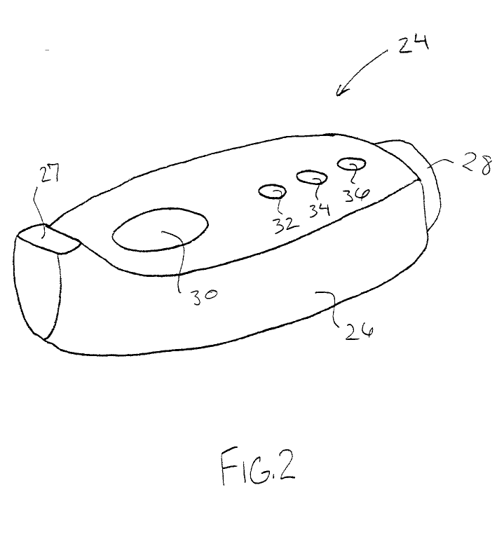

Shown in FIG. 2 is one embodiment of a drug delivery system 24 in the form of an MDI that may be used to aerosolize a metered amount of a drug formulation in a controlled manner, i.e. according to a specified dose regimen. However, it will be appreciated that the lockout mechanism may be incorporated into other aerosol delivery systems as previously described. System 24 comprises a housing 26 having a mouthpiece 27 through which a metered amount of a drug formulation may be supplied to a patient. Conveniently, housing 26 may be sized such that it will fit within a patient's hand. Housing may further be constructed of a lightweight, inexpensive material, such as ABS plastic to reduce costs and permit system 24 to be disposable. Reciprocally held within housing 26 is a canister 28 having a pressurized amount of drug formulation. Canister 28 is configured to aerosolize a metered amount of the drug formulation when canister 28 is depressed into housing 26 (and when dosing has been qualified as described hereinafter).

Held within housing 26 is a microcontroller, such as the PIC series, commercially available from Microchip Technologies, Inc., that is programmed with one or more dosing conditions that are based on a dosing regimen. The controller is configured to prevent dispensing of the drug formulation until the dosing conditions have been satisfied. Merely by way of example, a dosing regimen may require dosages to be administered over a certain number of weeks, with the number of dosages per day varying over the course of administration. The time between such doses may conveniently be referred to as inter-dose intervals. Further, certain time intervals, referred to as intra-dose intervals, may be required between each dosing time. For example, one dose may be administered over 10 minutes, with at least a two minute intra-dose interval between each inhalation. Hence, the controller may be configured to prevent dispensing of the drug formulation from canister 28 during the two minute intra-dose intervals as well as during the inter-dose intervals that occur throughout the day.

System 24 may also include an internal clock so that appropriate dispensing times may be determined. For example, system 24 may include a 32 KHz watch crystal that is electrically coupled to the microcontroller. Alternatively, system 24 may utilize the internal clock of the microcontroller to keep time. In one aspect, the clock may be configured to keep relative time. For example, the clock may begin keeping time upon insertion of canister 28 into housing 26. The clock will then keep time for 24 hour periods, with the clock being reset at the end of each period to avoid any cumulative error.

System 24 further includes a status button 30 that may be pressed by a user to determine if a dose has been qualified. Disposed on housing 26 are three visual indicators, such as LEDs 32, 34 and 36. Optionally, the visual indicators may have different colors that are representative of various states or conditions. For example, LED 32 may be green in color and will be lighted after button 30 is pressed and a dose has been qualified. If a dose has not been qualified, LED 34 (which may be yellow in color) is lighted indicating that the user must wait before the dose is qualified. LED 36 may be blue in color and will automatically light when canister 28 needs to be replaced, or when system 24 needs to be discarded.

If a dose has been qualified, the user simply places mouthpiece 27 into their mouth and depresses canister 28 into housing 26. System 24 uses a sensor that is coupled to the controller to sense the press. The controller then sends a signal to activate a lockout mechanism so that dosing may occur as described in greater detail hereinafter. Alternatively, once a dose has been qualified, the lockout mechanism may automatically be activated so that subsequent depression of canister 28 will cause a dose to be administered. System 26 may further include a dose counter that counts the dose so that system 26 will know when canister 28 needs to be replaced (and when to light LED 36). After canister 28 is depressed, the dosing clock is reset and begins counting until the next dose is qualified according to the schedule.

One feature of system 24 is that it requires two separate operations before a drug will be administered: pressing of status button 30 and pressing of canister 28. In this way, the risk of accidental child inhalation is minimized. Optionally, system 24 may include additional child lock out features as described hereinafter.

Housing 26 may conveniently be fabricated in two halves to facilitate the introduction of the various components into the housing at the time of manufacture. When placed together, the two halves define an internal cavity having an open end into which canister 28 is received. As shown in FIG. 2, when canister 28 is firmly seated within the cavity, canister 28 protrudes from the cavity to facilitate removal. Approximately halfway down the cavity is an O-ring that is trapped in place so that canister 28 will move relative to the O-ring when reciprocated within the cavity. The O-ring engages the canister in a snug fashion to minimize fluid ingress while allowing for canister displacement.

System 24 further includes a printed circuit board that is held within housing 26. The printed circuit board may include the microcontroller as well as various electrical components as described herein. System 24 may also include a canister switch that is employed to detect when canister 28 is inserted into housing 26. In one embodiment, the canister switch may be formed by a conductive surface on the O-ring that shorts a contact on the printed circuit board when displaced by canister 28.

The cavity of housing 26 may also include an opening at its bottom end for receiving the nozzle on canister 28. Another opening may also be provided for a dosing switch which engages the front of canister 28. When the user presses on the end of canister 28 for a dose, canister 28 slides a short distance within housing 26 until the dosing switch is closed. This closure is sensed by the electronics, and if the dose has been qualified, then canister 28 will be activated to deliver a dose of the drug formulation. In some embodiments, closure of the dosing switch may be used to count the number of times that canister 28 has been activated.

Although not shown, canister 28 includes a nozzle that is displaced a certain distance towards the canister body in order to deliver a metered amount of a substance. Canisters that are capable of dispensing a metered amount of a substance in this manner are known in the art and will not be described further. After the dose has been delivered, the nozzle is restored to its resting or home position before the next dose may be dispensed.

System 24 may use a variety of schemes to move the nozzle relative to the canister body. For example, system 24 may include an electromechanical actuator, such as a solenoid. The solenoid converts an electrical current into force and motion and may comprise a coil, a metal plunger, and a spring. When current flows through the coil, the resulting magnetic field causes the plunger to move with a force that distends the spring. When the current stops, the magnetic field collapses and the spring retracts the plunger. The solenoids used by the invention may be configured to be either push or pull, depending on current flow direction and spring configuration.

In one embodiment, the solenoid may be axially aligned with the canister nozzle. When the dosing switch is sensed, the solenoid is activated and the plunger is forced against the nozzle to cause activation. If the solenoid is not energized, then the plunger will not impede or press against the nozzle. Hence, with such a configuration, if a battery or electronics failure is experienced, the canister will not be activated.

In another embodiment, the solenoid may be employed to “arm” a mechanism the moment a dose has been qualified, or immediately after depression of canister 28 within housing 26 has been sensed. The armed mechanism may be configured to impede the travel of the nozzle while the user depresses canister 28 into housing 26. With such a configuration, the dosing switch would communicate to the electronics that the dose has been delivered because the canister has moved while the nozzle remained fixed. Such a configuration is advantageous in that the solenoid force (and hence power consumption) is determined by that required to arm the mechanism, rather than by displacing the nozzle. Hence, power consumption may be minimized.

As previously described, the electronics within system 24 may employ the use of a microcontroller. Conveniently, the microcontroller may be configured to operate down to about 2.5 volts. In this way, system 24 may operate using two alkaline batteries or a single lithium cell battery. As previously described, a small watch crystal may be employed to provide real-time operation to an accuracy of several seconds per day over a nominal temperature range. Conveniently, the microcontroller may activate the solenoid using a current drive transistor. The microcontroller may be configured to operate in an interrupt mode where any button or switch closure would wake up the microcontroller. Hence, power is needed only at certain times, thereby greatly minimizing power consumption and battery size.

System 24 may further include a normally open switch that would be used in connection with status button 30. Such a switch may be constructed by fabricating button 30 of an elastomer having a conductive pattern silk screened on its back side. When status button 30 is depressed, button 30 shorts a trace on the printed circuit board, thus indicating a closure. Optionally, the electronics may be waterproofed by conformal coating.

One particular advantage of such a configuration is that housing 26 may be constructed to be relatively small in size while still being able to hold all of the required components. For example, the required electronics may require only approximately 0.75 inches by 0.5 inches of printed circuit board space. Further, the necessary soldering may be only to the solenoid coil leads and the battery. Conveniently, the batteries may have metal tabs bonded to the electrodes at the time of manufacture for printed circuit board mounting.

At the time of assembly, system 24 is preferably tested, and the electronics placed into a sleep mode by closing the dosing switch and pressing status button 30 simultaneously. In the sleep mode, essentially zero battery drainage is experienced and system 24 will therefore have a shelf life as long as the battery chemistry permits. When the user inserts canister 28 into housing 26 for the first time, the canister switch is depressed, which wakes up the microcontroller. The controller sets the canister count to one and enables a one-second interrupt. The microcontroller then goes back to sleep so that no additional current is required. Once a second, the controller wakes up and increments the time counter. After a prescribed qualification, system 24 is placed into a qualified state. When the user presses status button 30, the microcontroller wakes up and lights LED 32 to indicate to the user that a dosage has been qualified. When a predetermined number of doses have been delivered, the microcontroller will flash LED 36 to indicate that the canister needs to be replaced. Canister removal and replacement is sensed by opening and closing of the canister switch. This mode of operation may continue until the required number of canisters have then been delivered. At this point, the microcontroller may light LED 36 to indicate that system 24 should be discarded. Alternatively, the microcontroller may activate the solenoid continuously until the battery is exhausted. This will prevent reuse and facilitate battery disposal.

Optionally, system 24 may include electronics to log data, such as the number of times status button 30 has been operated. This data may be stored in a low-cost, nonvolatile memory chip for later retrieval into a computer.

One advantage of system 24 is that it may be used to deliver a large number of doses using minimal power. Merely by way of example, when using a PIC16C54 microcontroller with a series S-63-38 tubular solenoid, commercially available from Magnetic Sensor Systems, up to 800 doses may be achieved when using a lithium MnO₂ cell battery or two alkaline AA batteries.

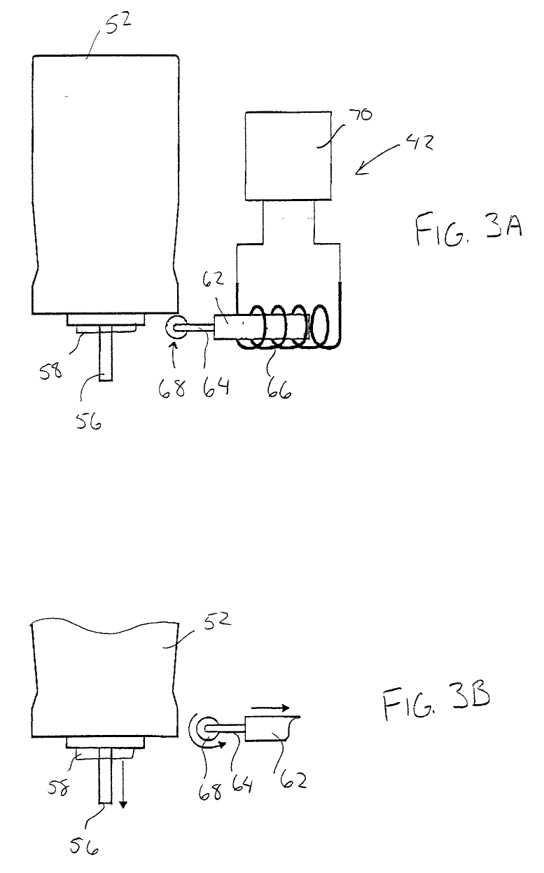

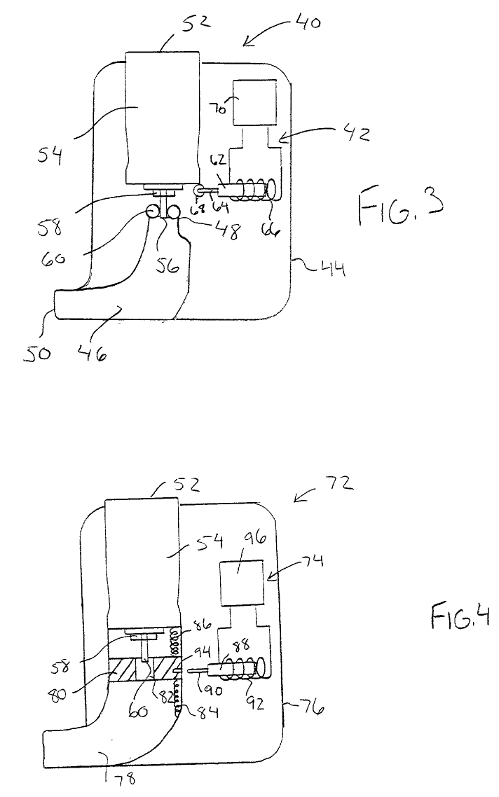

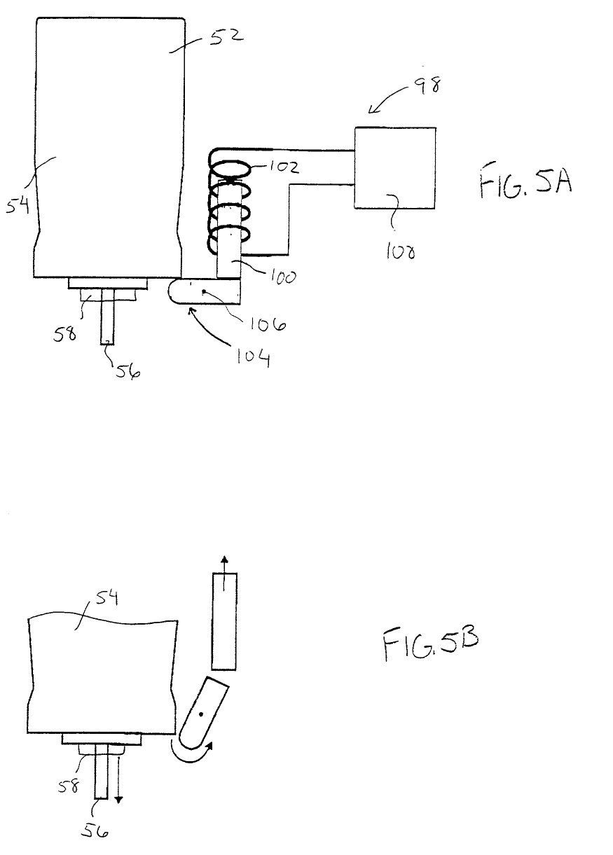

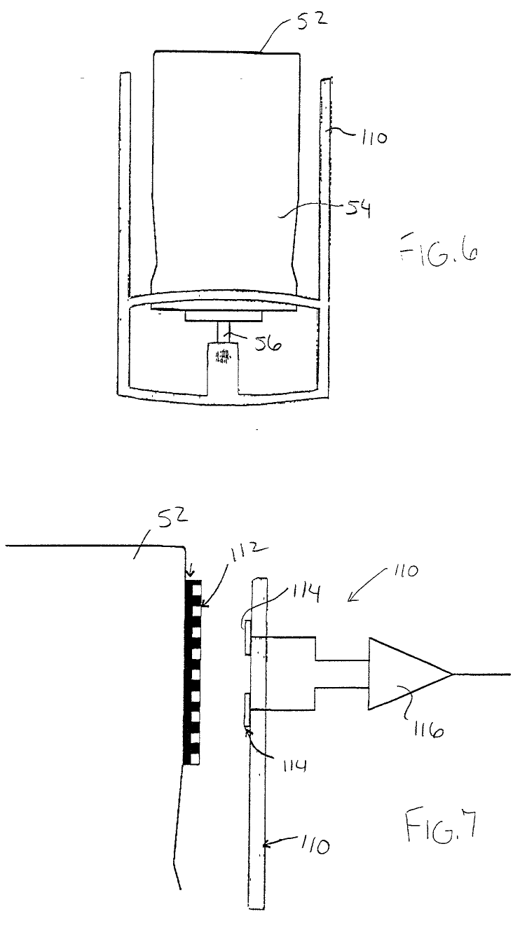

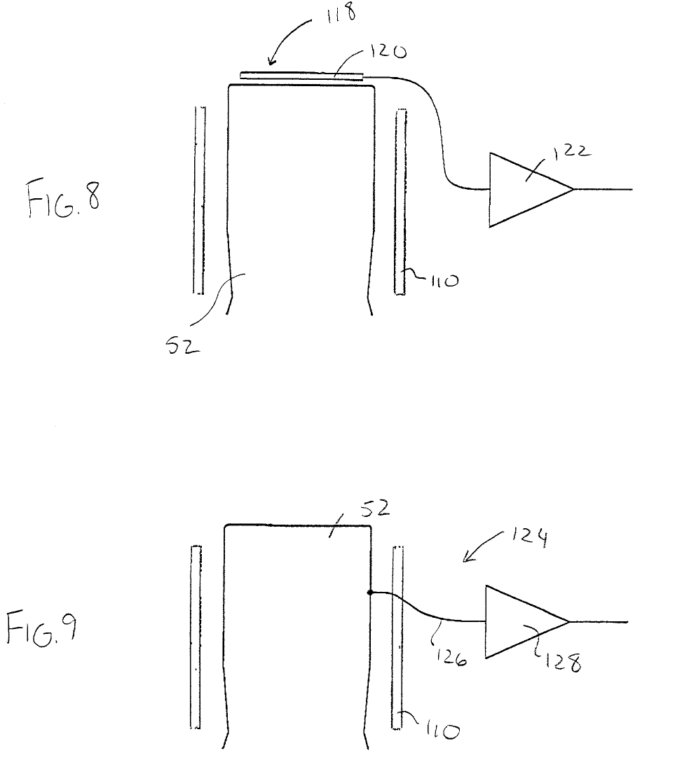

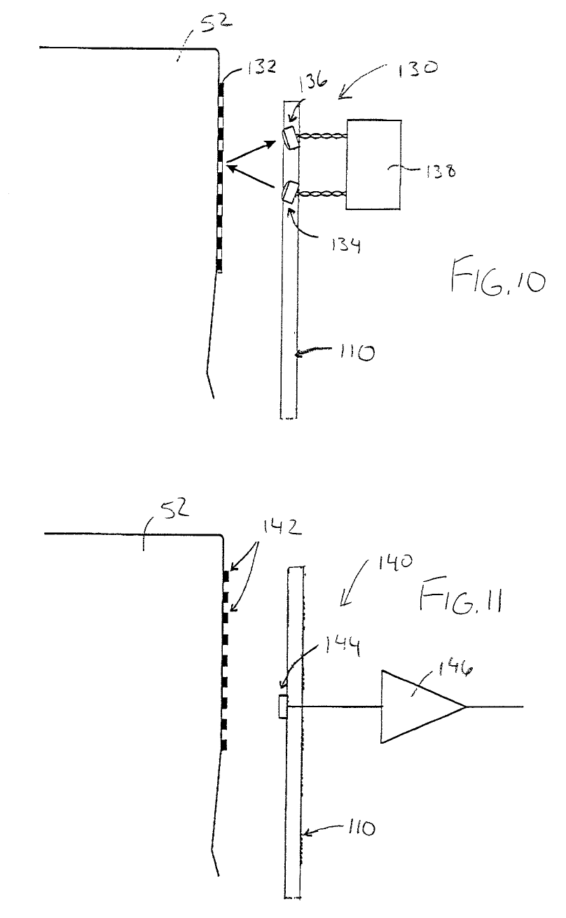

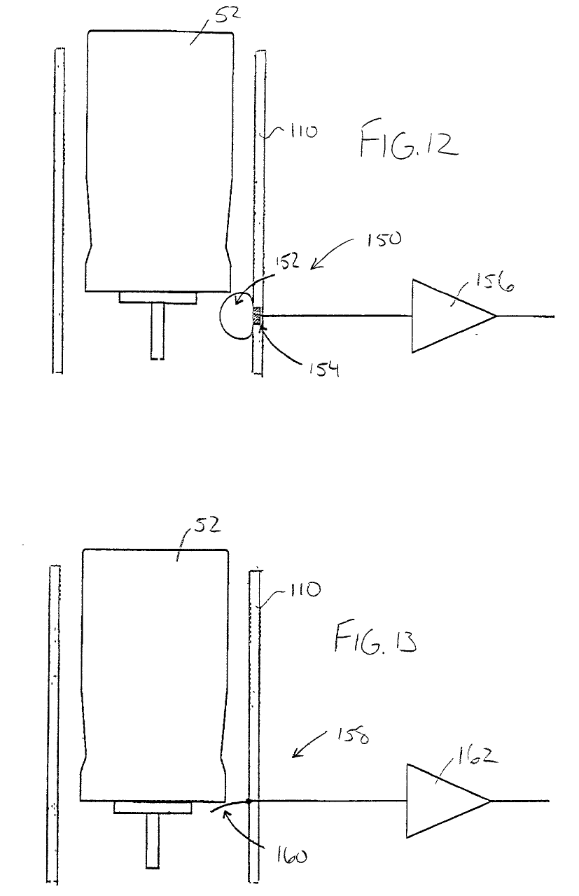

Referring now to FIG. 3, another embodiment of a drug delivery system 40 will be described schematically to illustrate the use of an electronic lockout device 42. In so doing, it will be appreciated that a similar lockout device may be included within drug delivery system 24 as previously described. System 40 comprises a housing 44 having a mouthpiece 46 with a first end 48 and a second end 50. Reciprocally disposed within housing 44 is a canister 52 that contains a pressurized drug formulation. Canister 52 comprises a canister body 54 and a nozzle 56 coupled to canister body 54. An actuator 58 is included on nozzle 56 to permit a metered amount of the drug formulation to be dispensed from nozzle 56 when actuator 58 is pressed toward canister body 54 as is known in the art.

Canister 52 is configured to extend above housing 44 to facilitate removal of canister 52 when empty. Canister 52 is further arranged so that nozzle 56 extends through first end 48 of mouthpiece 46. An O-ring 60 is disposed at first-end 48 and engages actuator 58 to dispense a metered amount of drug formulation when canister 52 is depressed into housing 44. Although not shown, system 40 further includes a biasing member, such as a spring, to return canister 52 to its home position after a dose of the drug formulation has been dispensed.

As also shown in FIG. 3A, electronic lockout device 42 comprises a solenoid 62 having a plunger 64. A coil 66 is provided to cause plunger 64 to retract when current is passed through coil 66. A spring (not shown) is held within solenoid 62 to maintain plunger 64 in an extended position as shown in FIGS. 3 and 3A. In the extended position, a roller 68 on plunger 64 engages canister 52 to prevent depression into housing 44. In this way, no current is required by solenoid 62 to maintain plunger 64 in the extended position. As such, canister actuation is prevented in the absence of any supplied power, i.e., when device 42 is in the inactive state. When electric current is supplied to coil 66, plunger 64 retracts to permit canister 52 to be depressed and to allow a metered amount of the drug formulation to be dispensed into mouthpiece 46 where it may be inhaled through second end 50.

Coil 66 is electrically connected to a printed circuit board 70 that may include a microcontroller and a clock in a manner similar to that previously described in connection with delivery system 24. In this way, a user may request to receive a dose by pressing a status button in a manner similar to that described in connection with drug delivery system 24. If a dose has been qualified, the microcontroller on circuit board 70 may send a signal to cause electrical current to pass through coil 66, thereby causing plunger 64 to retract as shown in FIG. 3B. The user may then press canister 52 into housing 44 to release a metered amount of the drug formulation. A sensor (not shown) may be employed to detect when canister 52 has been depressed to record that a dose has been dispensed. Further, the sensor may be able to detect when canister 52 has returned to the home position so that solenoid 62 may return to its inactive state where plunger 64 will engage canister 52 and prevent its depression into housing 44. In an alternative aspect, such a sensor may also be used by the microcontroller to activate solenoid 62 after a dose has been qualified and after the user begins to press canister 52. In this way, solenoid 62 will be activated only when the user presses the status button and then presses canister 52 into housing 44.

Shown in FIG. 4 is another embodiment of a drug delivery system 72 having an electronic lockout device 74. As with lockout device 42, it will be appreciated that a lockout device similar to lockout device 74 may be used with drug delivery system 24. System 72 comprises a housing 76 having a mouthpiece 78. A canister is reciprocally disposed within housing 76 in a manner similar to drug delivery system 40. For convenience of discussion, the canister is labeled with the same reference numerals used in connection with drug delivery system 40. Disposed below canister 52 is a stop 80 having an opening 82 through which nozzle 56 extends. In this way, a dose delivered from canister 52 is permitted to pass into mouthpiece 78 where it may be inhaled by the patient. Stop 80 is reciprocally held within housing 76 and is biased upward by a spring 84. Also positioned between stop 80 and canister 52 is a spring 86. In this way, when lockout device 74 is in the inactive state as shown in FIG. 4, canister 52 may be depressed into housing 76. In so doing, spring 86 will force stop 80 downward so that actuator 58 will not engage stop 50. Hence, when lockout device 74 is in the inactive state and canister 52 is depressed, a dose of the drug formulation is prevented from being dispensed. When canister 52 is released, spring 84 will move stop 80 and canister 50 to their home position.

Lockout device 74 comprises a solenoid 88 having a plunger 90 and a coil 92. When electrical current is supplied to coil 92, plunger 90 is moved to a dose-preventing position where it enters an opening 94 in stop 80. In the dose-preventing position, plunger 90 prevents movement of stop 80 within housing 76. Hence, when canister 52 is depressed, actuator 58 will engage stop 80 to dispense a metered amount of the drug formulation into mouthpiece 78. When electrical current is stopped, plunger 90 will retract to the position shown in FIG. 4.

Coil 92 is electrically coupled to a printed circuit board 96 having a microcontroller similar to that previously described in connection with FIG. 3. Hence, once the microcontroller determines that a dose has been qualified, a signal may be sent to supply current to coil 92 to place device 74 in the active state where a dose of the drug formulation may be dispensed. Optionally, a sensor may be employed to sense when canister 52 is depressed, with the microcontroller using the signal to cause electrical current to be supplied to coil 92. Further, such a sensor may be employed to count the number of doses delivered by canister 52.

Referring now to FIGS. 5A and 5B, an alternative lockout device 98 that may be used with drug delivery system 40 will be described. Lockout device 98 comprises a solenoid 100 having a coil 102. A lever arm 104 is positioned below solenoid 100 and is pivotally attached to the system housing at a pivot point 106. Arm 104 extends below canister 52 when lockout device 98 is in the inactive state to prevent depression of canister 52 into the housing.

Lockout device 98 further comprises a circuit board 108 having a microcontroller that is employed to send a signal to have electrical current supplied to coil 102 to place lockout device 98 in the active state. In so doing, solenoid 100 is moved upward as shown in FIG. 5B. In this way, solenoid 100 becomes disengaged from arm 104 which is free to pivot when canister 52 is pressed downward. As canister 52 is pressed downward, actuator 58 is engaged to dispense a metered dose of the drug formulation in a manner similar to that previously described.

FIG. 6 schematically illustrates canister 52 when disposed within a housing 110. Housing 110 represents the basic structure employed to properly position canister 52 within a drug dispensing device. For convenience of illustration, housing 110 is shown with the various detection systems of FIGS. 7-15 that will be described hereinafter. However, it will be appreciated that the various detection systems may be used in connection with other types of housings, and the invention is not intended to be limited to the specific configuration shown in FIG. 6.

As previously described, the drug delivery systems of the invention may be provided with sensors or detectors to indicate when a canister has been inserted into the device and/or when the canister has been depressed to dispense a dose of the drug formulation. FIGS. 7-13 illustrate various detection systems that may be employed to detect when canister 52 has been depressed in order to deliver a dose of the drug formulation and/or when canister 52 has been inserted into the housing.

In FIG. 7, a capacitive detection system 110 comprises an isolation strip 112 having alternating conductive and nonconductive areas that is attached to canister 52. One or more sensing electrodes 114 are coupled to the inner wall of housing 110 to detect the passage of these areas. Electrodes 114 are coupled to a sensing amplifier 116 to permit the signals to be transmitted to a printed circuit board having a microcontroller in a manner similar to that previously described.

As canister 52 is moved within housing 110, electrodes 114 sense a change in capacitance as the alternating conducting and nonconducting areas on strip 112 pass the electrodes. Hence, detection system 110 may be employed to detect when canister 52 is inserted into housing 110 and when canister 52 has been actuated. One advantage of detection system 110 is that it may be employed to detect both the rate and extent of canister movement.

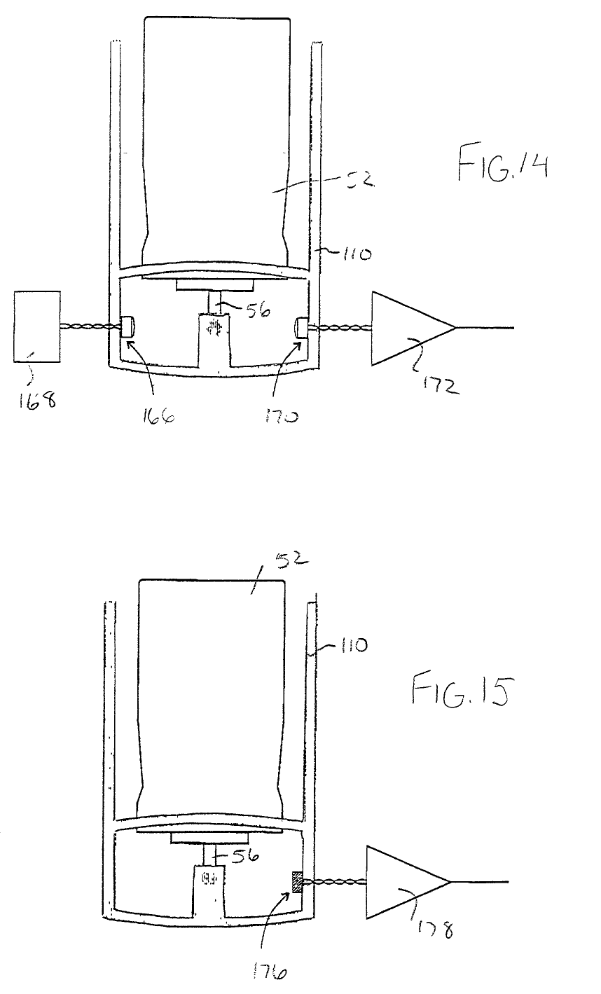

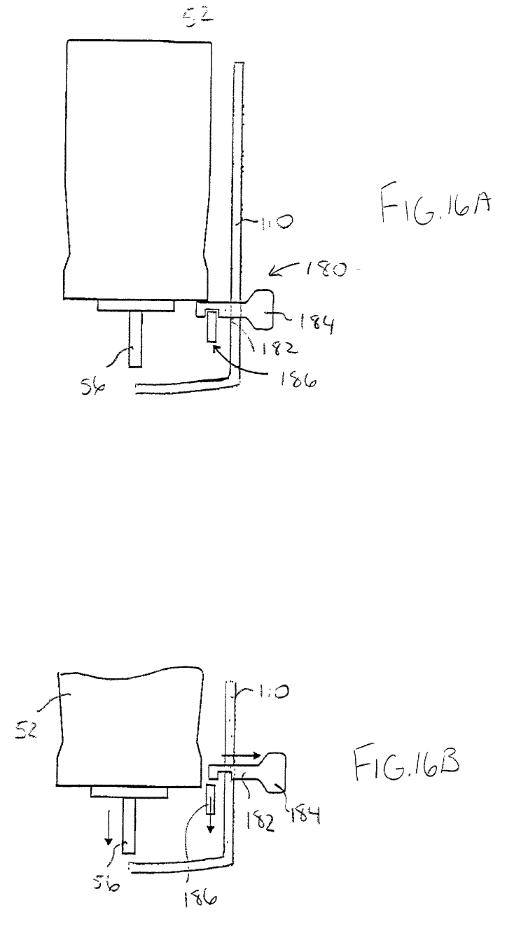

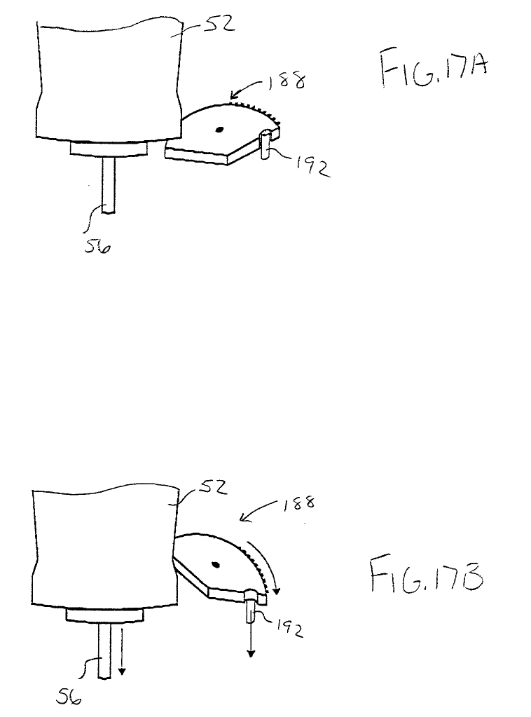

FIG. 8 illustrates a detection system 118 that comprises a thin membrane switch 120 that is coupled to a logic detect 122. To depress canister 52, the user must depress membrane switch 120 which sends a signal to logic detect 122 to indicate to the microcontroller that canister 52 has been depressed. Membrane switch 120 may be employed to detect both the occurrence of the depression and the duration of the depression.

FIG. 9 schematically illustrates a touch-sensitive detection system that comprises an electrical conductor 126 that is coupled to canister 52 and to a touch-sensitive amplifier 128. If canister 52 is constructed of metal, touching of canister 152 will send a signal to amplifier 128 to indicate that canister 52 has been pressed.

FIG. 10 illustrates an optical detection system 130 that comprises a strip 132 having alternating reflecting and nonreflecting areas that is attached to canister 52. System 130 further includes a light source 134, such as an LED, that is directed toward the strip 132 in such a way that its reflected light may be detected by a detector 136. Light source 134 and light detector 136 are coupled to source/detector electronics 138 that are used to activate light source 134 and to operate detector 136. The detected signals may then be sent to a microcontroller for processing. Detection system 130 is advantageous in that it may detect both event and duration of actual canister movement.

FIG. 11 illustrates a magnetic detection system 140 that comprises a plurality of spaced apart magnets 142 that are coupled to canister 52. For example, magnets 142 may comprise thin, flexible rubber magnets that are equally spaced. A magnetic sensor 144, such as a Hall effect sensor, may be coupled to housing 110 to detect both event and duration of actual canister movement. A sensing amplifier 146 is employed to amplify the sensed signals and transmit them to a microcontroller.

FIG. 12 illustrates a pressure detection system 150 that comprises an inflatable bladder 152, such as a rubber bladder, that is filled with a gas, such as air, and fitted with a pressure transducer 154. When canister 52 is depressed, canister 52 engages bladder 152 to increase the pressure. This change is sensed by transducer 154, with the signal being amplified by an amplifier 156 so that an appropriate signal may be sent to a microcontroller. Transducer 154 is able to detect both event and duration of canister movement.

FIG. 13 illustrates a piezoelectric detection system 158 that comprises a thin flexible piezoelectric film 160 that is coupled to housing 110. Film 160 is positioned near the edge of canister 52 and is deflected when canister 52 is depressed. The resulting signal is amplified by an amplifier 162 and sent to a microcontroller. Detection system 158 is able to detect both the event of and the duration of canister movement.

As an alternative to detecting canister movement as an indicator of dose delivery, detection systems may be employed to detect when a dose has been transferred from nozzle 56. Examples of such detection systems are illustrated in FIGS. 14 and 15.

FIG. 14 illustrates an optical detection system 164 that comprises a light source 166 that is coupled to source electronics 168 that are employed to produce light at light source 166. A light detector 170 is disposed at an opposite side of housing 110 such that the line of sight between light source 166 and light detector 170 is intercepted by the emitted dose from nozzle 56. The output of light detector 170 is diminished when the spray obscures the beam from light source 166. This signal is amplified by an amplifier 172 and is transferred to a microcontroller.

FIG. 15 illustrates an acoustic detection system 174 that comprises a microphone 176 that is coupled to housing 110. Microphone 176 is situated so that it does not interfere with the spray from canister 56 while still being able to detect its presence by virtue of the associated pressure change. Alternatively, a pressure-sensitive transducer may be employed instead of a microphone. An amplifier 178 is employed to amplify the signal and to transmit it to a microcontroller.

As previously described, actuation of the canister may be prevented by use of a lockout device having an electrically activated solenoid and plunger. However, it will be appreciated that other lockout mechanisms may be utilized. For example, shape memory alloys, such as Nitinol, may be employed in a variety of configurations to prevent actuation. Shape memory alloys contract when heated, such as when applying an electrical pulse to the material. The shape memory alloys may be configured in a variety of shapes and forms to act as a lockout mechanism. Merely by way of example, the lockout mechanism may be in the form of a wire, a lever, a spring, a tube and the like. For instance, a tube may be employed in lockout systems configured similar to that previously described in connection with FIG. 3. When heated, the shape memory alloy contracts to move the lockout mechanism to the active state where dispensing may occur. As another example, a curved member may be provided that is configured to straighten when activated. Such a curved member may be used in a system similar to that described in connection with FIGS. 5A and 5B.

The current or voltage pulse applied to the shape memory alloy may be a rapidly rising square pulse or a slower ramp. Alternatively, many short pulses may be delivered in rapid succession as a pulse train. Contraction may be accomplished in about 100 ms. The electrical requirements and the force developed may depend on the size of the shape memory alloy member.

The drug delivery systems of the invention may optionally include “child proof” lockout mechanisms that require an additional level of manual dexterity and user intervention before canister actuation is permitted. Two such examples are illustrated in FIGS. 16A and 16B, and in FIGS. 17A and 17B.

FIGS. 16A and 16B illustrate a lockout mechanism 180 that comprises a rigid member 182, having a pull tab 184 that protrudes from housing 110. Member 182 is held in place by a locking pin 186. When locking pin 186 is in a locked position, member 182 prevents downward movement of canister 52. At dosing time, pin 186 may be withdrawn by a solenoid, shape memory alloy member, or the like. Pull tab 184 must then be withdrawn in a timely manner to allow canister travel and actuation. Alternatively, pull tab 184 may be configured to require more complex maneuvering, such as twisting, turning, deflecting, pushing, or the like.

With such a configuration, a child is prevented from operating the system at dosing time without first comprehending lockout mechanism 180 and performing the maneuver within an allowed time window as dictated by the microcontroller. Furthermore, lockout mechanism 188 provides a fail-safe feature whereby failure of the electronics or battery will result in a permanently locked out device.

FIGS. 17A and 17B illustrate a lockout mechanism 188 that comprises a rigid disk 190 that protrudes from the housing and serves as a thumb wheel. When in the locking position as shown in FIG. 17A, disk 190 prevents downward travel of canister 52. At dosing time, a pin 192 is withdrawn as shown in FIG. 17B by use of a solenoid, shape memory alloy member, or the like. The user must then rotate disk 190 within a given time window to clear a path to permit canister 52 to be depressed to deliver the dose.

Hence, the various lockout mechanisms described herein may be used to interfere with any moving parts to prevent delivery of a drug formulation. For example, the lockout mechanisms may interfere with movement of parts required to release a pressurized drug formulation from a canister. As another example, the lockout mechanisms may be used to prevent the puncture of a receptacle that contains a dry powder drug formulation, such as those described in U.S. Pat. Nos. 5,785,049 and 5,740,794, and in U.S. application Ser. Nos. 09/004,558 and 60/141,793, previously incorporated by reference.

According to a particularly preferred embodiment of the present invention, the lockout systems of the invention may also find use with aerosol delivery devices to administer nicotine to provide a smoking cessation therapy. For example, metered dose inhalers provided with the lockout feature of this invention may be used to administer a nicotine formulation for smoking cessation treatment. Nicotine formulations for administration by such devices may be produced according to the methods disclosed in U.S. patent application Ser. No. 09/218,212, previously incorporated by reference.

Nicotine formulations according to his embodiment comprise nicotine base, or a pharmaceutically acceptable acid addition salt of nicotine, such as nicotine bitartrate. Preferred formulations comprise a surfactant, preferably a phospholipid. Other excipients are known in the art, including those disclosed in the patents and patent applications cited above. Preferred excipients include, but are not limited to sugars such as lactose, buffering agents such as sodium phosphate, and calcium chloride.

A particularly preferred nicotine composition according to this invention comprise a fine particle fraction and fine particle dose of at least 70%. The fine particle fraction is the ratio of particles deposited in stages 2 through 7 to those deposited in all stages of an Anderson Cascade Impactor while the fine particle dose is the percentage of a unit dose of powder delivered to a patient. The nicotine formulations are preferably produced by spray drying processes known in the art in order to produce the particulate compositions of the present invention.

The delivery devices of the invention may be programmed to actuate the lockout mechanisms to permit dosing according to a specified dosing schedule. Merely by way of example, for smoking cessation therapy the device may be programmed to specify a lockout period between dosing intervals that changes over a 10-week, 3-step program, as well as a lockout at actual dosing time, with one dose (three puffs) administered over ten minutes with at least a two minute interval between each puff.

As previously described, the delivery devices of the invention may include an electronic timer and a lockout mechanism programmed to regulate both intra-dose puffs and inter-dose intervals. For example, a user may press a status button on the device to see if the next dose has been qualified. If so, a visual signal is produced and a canister is pressed to release a dose. The device then counts the dose and resets the dosage clock and waits until the next dose is ready according to the schedule.

The invention has now been described in detail for purposes of clarity and understanding. However, it will be appreciated that certain changes and modifications may be practiced within the scope of the appended claims.

What is claimed is:

1. A hand-held, portable, aerosol drug delivery system, comprising:
a disposable container containing a drug formulation;
an aerosol generator for aerosolizing the drug formulation;
a prevention device which prevents access to the drug formulation when in an inactive state and which permits access to the drug formulation when in an activated state.

2. A system as in claim 1, wherein the prevention device comprises an electronic lockout device having a lockout element that is positioned in a dose preventing position when in the inactive state, and is movable to a dosing permitting position when electric current is supplied to place the lockout device in the activated state.

3. A system as in claim 2, wherein the lockout device further comprises circuitry for supplying electrical current to move the lockout element to the dose permitting position when the lockout device is in the activated state.

4. A system as in claim 2, wherein the lockout device further comprises a controller having an associated memory for storing a dosing condition, and wherein the controller is configured to send a signal to place the lockout device in the activated state only after the dosing condition has been satisfied.

5. A system as in claim 2, wherein the container comprises a canister, and wherein the aerosol generator comprises a metering valve and an actuator operably coupled to the canister.

6. A system as in claim 5, further comprising a housing, wherein the canister is reciprocally held within at least a portion of the housing between a home position and a dosing position where the actuator is engaged to open the metering valve and to permit the escape of a metered amount of the drug formulation from the canister.

7. A system as in claim 6, wherein the lockout element is positioned to prevent engagement of the actuator when in the dose preventing position to thereby prevent opening of the metering valve.

8. A system as in claim 7, wherein the lockout element has a distal end that is engageable with the canister to prevent substantial displacement of the canister into the housing when the lockout element is in the dose preventing position.

9. A system as in claim 8, wherein upon placement of the preventing device into the activated state, the distal end of the lockout element is retracted to permit displacement of the canister into the housing and to permit engagement of the actuator to open the metering valve.

10. A system as in claim 7, wherein the canister is movable within the housing when the preventing device is in the inactive state, and further comprising a stop that is reciprocally disposed within the housing below the actuator, and wherein the lockout element has a distal end that is engageable with the stop when in the activated state to prevent movement of the stop within the housing such that displacement of the canister engages the actuator with the stop to permit dispensing of the metered drug formulation when the preventing device is in the activated state.

11. A system as in claim 1, further comprising a high pressure gas source to assist in aerosolizing the drug formulation when the preventing device is in the activated state.

12. A system as in claim 1, further comprising a dose counter disposed to count the number of doses of the drug formulation dispensed from the container.

13. A system as in claim 12, wherein the container is reciprocatably disposed within a housing, and wherein the dose counter comprises a dose counting circuit positioned to sense when the container has been reciprocated within the housing.

14. A system as in claim 13, wherein the dose counter further comprises a display for indicating if the container contains an amount of drug formulation.

15. A system as in claim 5, further comprising a nozzle operable coupled to the canister, and wherein the housing further includes a mouthpiece disposed to receive the drug formulation from the nozzle.

16. A system as in claim 15, wherein the mouthpiece has a first end and a second end, and wherein the nozzle is positionable within an opening adjacent the first end of the mouthpiece to permit the aerosolized drug formulation to be delivered to a patient upon inhalation through the second end of the mouthpiece.

17. A method for administering a drug formulation, the method comprising:
providing a container having an amount of a drug formulation;
preventing the transfer of the drug formulation from the container with an electronic lockout device when the lockout device is in an inactive state; and
supplying electrical current to the lockout device to place the lockout device in an active state, thereby permitting the transfer of the drug formulation from the container.

18. A method as in claim 17, wherein the electronic lockout device comprises a lockout element that is positioned in a dose preventing position when in the inactive state, and further comprising moving the lockout element to a dosing permitting position when electric current is supplied to place the lockout device in the activated state.

19. A method as in claim 18, wherein the container comprises a canister having a metering valve and an actuator, wherein the canister is reciprocatably held within a housing between a home position and a dosing position, and further comprising depressing the canister into the housing to the dosing position to engage the actuator and to release a metered amount of the drug formulation when the lockout device is in the active state.

20. A method as in claim 19, further comprising preventing engagement of the actuator when the lockout element is in the dose preventing position.

21. A method as in claim 20, further comprising engaging the canister with the lockout element to prevent movement of the canister to the dispensing position when the lockout element is in the dose preventing position.

22. A method as in claim 21, further comprising disengaging the lockout element from the canister to permit movement of the canister to the dispensing position upon supply of the electrical current.

23. A method as in claim 20, further comprising engaging the lockout element with a stop that is positioned below the actuator upon supply of the electrical current, and further comprising depressing the canister into the housing to engage the actuator with the stop.

24. A method as in claim 18, further comprising stopping the supply of the electric current to the lockout device after the drug formulation has been transferred from the container.

25. A method as in claim 24, further comprising supplying electric current to the lockout device to permit another dosing only after a certain dosing conditions have been satisfied.

26. A method as in claim 25, further comprising counting the number doses transferred from the container.

27. A method as in claim 26, further comprising displaying whether the container contains an amount of drug formulation based on the number of counts.

28. A hand-held, portable, aerosol drug delivery system, comprising:
a housing having a mouthpiece;
a canister that is movable within the housing when manually depressed into the housing, the canister having a metering valve that is operable to release a metered amount of a drug formulation from the canister; and
a control system to control opening of the valve such that the valve is only opened when a force is manually applied to depress the canister into the housing and when a dosing condition has been satisfied.

29. A system as in claim 28, wherein the control system comprises a controller and a locking mechanism, wherein the controller is configured to send a signal to the locking mechanism to permit opening of the valve once the dosing condition has been satisfied.

30. A system as in claim 29, wherein the dosing condition is the passage of a certain amount of time between dosings, and further comprising an electronic clock coupled to the controller to measure the passage of time between dosings.

31. A system as in claim 28, wherein the locking mechanism is normally in a dose preventing position and is movable to a dosing position when electrical current is supplied to the locking mechanism to permit opening of the valve when the canister is depressed.

32. A system as in claim 28, wherein the locking mechanism includes a locking element that engages the canister to prevent depression of the canister into the housing when in the dose preventing position.

33. A system as in claim 28, wherein the canister includes an actuator, and wherein the locking mechanism includes a locking element that engages a stop that in turn engages the actuator when in the dose permitting position and when the canister is depressed into the housing.

34. A method for administering a nicotine formulation for smoking cessation therapy, the method comprising:
providing an amount of a nicotine formulation;
preventing the aerosolization of the nicotine formulation with a lockout device when the lockout device is in an inactive state;
supplying electric current to the lockout device to place the lockout device in an active state; and
aerosolizing the nicotine formulation.

35. A method as in claim 34, further comprising controlling when electric current may be supplied to the lockout device based on a specified dosing schedule.

36. A system as in claim 1, wherein the drug formulation comprises nicotine.
